(19) United States
(12) Patent Application Publication
(10) Pub. No.: US 2024/0384289 A1
(43) Pub. Date: Nov. 21, 2024

(54) Lettuce Plants with Bremia-Resistance Providing Genomic Fragments from Lactuca Serriola

(71) Applicant: Bejo Zaden B.V., Warmenhuizen (NL)

(72) Inventors: Stefanus Johannes Kaandorp, Warmenhuizen (NL); Claudia Jacoba Wilhelmina Christianen, Warmenhuizen (NL); Teunis Scheurwater, Warmenhuizen (NL); Roelof Marinus Veenstra, Warmenhuizen (NL); Albertus Johannes Maria Schrijver, Warmenhuizen (NL)

(21) Appl. No.: 18/696,399

(22) PCT Filed: Sep. 29, 2021
(86) PCT No.: PCT/EP2021/076790

§ 371 Date: Mar. 28, 2024

(51) Int. Cl. C12N 15/82; C12Q 1/6895

(52) U.S. Cl. CPC C12N 15/8279; C12Q 1/6895; C12Q 2600/13; C12Q 2600/156

(57) ABSTRACT

The present invention relates to lettuce plants with one or more Bremia-resistance providing genomic fragments from Lactuca serriola. The present invention further relates to methods for identifying Bremia resistant lettuce plants, methods for providing Bremia-resistant lettuce plants with and means for identifying Bremia-resistant lettuce plants. Specifically, the present invention relates to lettuce plants resistant to Bremia, the lettuce plants includes a first and a second Bremia-resistance providing genomic fragment from Lactuca serriola, a third and a fourth Bremia-resistance providing genomic fragment from Lactuca serriola, a fifth Bremia-resistance providing genomic fragment from Lactuca serriola, or combinations thereof.

### CROSS-REFERENCE TO RELATED APPLICATION

This application is the United States national phase of International Application No. PCT/EP2021/076790 filed Sep. 29, 2021, the disclosure of which is hereby incorporated by reference in its entirety.

### REFERENCE TO A SEQUENCE LISTING

The Sequence Listing associated with this application is filed in electronic format via Patent Center and is hereby incorporated by reference into the specification in its entirety. The name of the text file containing the Sequence Listing is 2401365_ST25.txt. The size of the file is 11,780 bytes, and the file was created on Mar. 6, 2024.

### BACKGROUND OF THE INVENTION

### Field of the Invention

The present invention relates to lettuce plants with one or more Bremia-resistance providing genomic fragments from Lactuca serriola. The present invention further relates to methods for identifying Bremia resistant lettuce plants, methods for providing Bremia-resistant lettuce plants and means for identifying Bremia-resistant lettuce plants.

### Description of Related Art

Lettuce, generally designated as Lactuca sativa, is a cultivated plant that belongs to the daisy family (Asteraceae). This family includes several other food crops such as chicory, endive, sunflower, globe artichoke or Jerusalem artichoke also called topinambour. The Asteraceae family with over 23 000 accepted species names has a worldwide distribution from the tropics to the polar regions. It is of economic importance in agriculture due to the fact that plants from this family are a source of cooking oils, sunflower seeds and food crops. Plants from the daisy family are also used in horticulture with daisies, dahlias or chrysanthemums as examples.

Within the genus Lactuca, the most important food crop is lettuce but also other species are known such as L. serriola, L. virosa and L. saligna. Although these other species have often undesirable agricultural qualities as compared to lettuce such as: bitter flavor, latex content or presence of leaf spines, they often carry genetically-encoded plant-pathogen resistances that can be transferred to lettuce by introgression or genetic modification.

Lettuce is an annual crop mostly grown as a leaf vegetable. Its native range spreads from the Mediterranean to Siberia but due to its use in agriculture it can now be found all over the world. Lettuce produces crispy leaves and has an average size of 15-30 cm (e.g., iceberg, butterhead, babyleaf). The leaves are most often green and sometimes red but other colors are known. There is also a rather wide diversity in leaf shapes and textures. Breeding efforts have resulted in different types of lettuce, which include: leaf lettuce, batavia, iceberg also known as crisphead, romaine, butterhead or summer crisp and also stem, and oilseed. Lettuce is grown on a small scale as well as on a largescale and is often sold to wholesale salad companies.

The original markets for large-scale lettuce production were Western Europe and Northern America but China is an important market now as well. Generally, as compared to other vegetables, lettuce does not store well. However, thanks to improvements in packaging techniques the shelf life can now be prolonged which contributes to the increase of lettuce in the market share of vegetable production.

Lettuce has been known since ancient times and it was first used for its seeds and oils and only later bred for its leaves. Lettuce is eaten either raw or cooked. The most common way of eating lettuce is in salads but it is also added to soups, wraps, sandwiches or stir-fry dishes.

Lettuce is generally considered a healthy food by consumers. With over 95% of its fresh weight being water, it is low in calories and is the food of choice in diets. Lettuce is a source of vitamin K, vitamin A and vitamin A provitamins, folate, iron, fiber and to a lesser extent of other vitamins and microelements.

A real threat to growing lettuce is plant pathogens. They can significantly reduce yield and affected plants are visually less appealing to the consumer, both factors lead to significant financial loss for the producers. Pathogens attacking lettuce can be of bacterial origin (e.g. Rhizorhapis suberifaciens, Xanthomonas campestris), viruses (e.g. Lettuce Mosaic Virus, Tomato Spotted Wilt Virus), eukaryotic microorganisms (Bremia lactucae, Septoria lactucae, Sclerotinia sclerotiorum) nematodes (Meloidogyne incognita) and insects (Aphids, Thrips).

Downy mildew in lettuce is a disease caused by the oomycete Bremia lactucae. Other members of the phylum oomycota include Pythium and Phytophthora. Bremia lactucae is host-specific and is a highly variable pathogen rapidly breaking resistance.

Bremia lactucae occurs worldwide and poses a significant hazard to both yield and quality of lettuce. The oomycete can infect the lettuce plant at any stage of growth, after which the first symptoms of downy mildew become visible as chlorotic yellow spots on the leaf surface. Within 24-48 hours white sporulation becomes visible on the lower side of the leaf. During the infection the lesions become increasingly larger and more chlorotic until the leaves become completely brown. Typically, sporulation only occurs, when lettuce is susceptible to Bremia lactucae.

One possible way to combat plant pathogens is to use pesticides. This approach has many disadvantages including: negative impact on the natural environment, often low selectivity and costs resulting from labor intensity. The use of pesticides is also becoming more and more restrained by law. It is not allowed to use chemical pesticides in organic food production. There is a high demand for organic food from consumers and its market share is growing, making it a product of increasing importance.

The reduction of yield and resulting financial consequences together with the aforementioned reasons for avoiding pesticides have served as an incentive for lettuce breeders to develop plants that have genetically encoded resistance to the pathogen.

Resistance breaking isolates occur frequently through mutation of the virulence genes during spore formation preceding the propagation of Bremia lactucae. When resistance-breaking isolates persist over multiple years and break important commercial resistance genes the International Bremia Evaluation Board (IBEB) can nominate such resistance-breaking isolates as new races. Currently there are over 20 relevant official races known for Bremia lactucae (B1: 16EU to B1: 37EU).

Within the genus Lactucae, to which lettuce belongs, there are different species which are resistant to Bremia lactucae. The resistance is generally based on dominant genes, known as Dm-resistance genes (Dm stands for Downy mildew). The resistance mechanism can work according to the gene-for-gene model based on the specific interaction between product(s) of the plant-specific resistance gene and the pathogen-specific avirulence gene which results in resistance of the lettuce plant. The R-genes encode for extracellular proteins with a nucleotide binding site (NBS), fused to a leucine-rich repeat (LRR) with different N-terminal domains (Toll/Interleukin-1 Receptor homology TIR or coiled-coil motif CC). To date, R-genes are grouped in 4/5 classes based on the conserved domain organization. In other cases Dm resistance is based on multiple genes (QTLs).

Due to the high variability of the pathogen, which is attributed to the occurrence of frequent mutations in avirulence genes, the race-specific resistance mediated by the various Dm resistance genes is usually rapidly broken by newly emerging races of the Bremia lactucae pathogen.

The prior art discloses lettuce plants where a genetically encoded resistance against Bremia lactucae is introduced into cultivated plants from wild relatives, most often from L. saligna (WO2011003783, WO2015136085, WO2009111627) but also from L. virosa (WO2000063432). The genetically encoded resistance in lettuce plants provides resistance to specific races of Bremia lactucae.

As mentioned, there is a rapid adaptation of Bremia to the genetically encoded resistance. New Bremia isolates emerge that can evade the recognition by the host and thus overcome this race-specific resistance. To counter this adaptation breeders combine or stack resistances, i.e. introduce two or more different Bremia resistance genes into one plant so as to increase the chance of a plant to overcome Bremia infection or reduce the probability that the pathogen overcomes the resistance. There is thus a need in the field to provide new and alternative genetically encoded resistances against Bremia to enable the breeders to develop novel lettuce cultivars that are resistant to Bremia.

### SUMMARY OF THE INVENTION

It is an object of the present invention, amongst other objects, to meet the above need in the art.

This object, amongst other objects, is met by the present invention as outlined in the appended claims.

Specifically, the above object, amongst other objects is met, according to a first aspect, by providing lettuce plants resistant to Bremia, the lettuce plants comprise a first and a second Bremia-resistance providing genomic fragment from Lactuca serriola, wherein the first Bremia-resistance providing genomic fragment is located on chromosome 4 of the lettuce plants and comprises a nucleic acid sequence selected from the group consisting of SEQ ID No. 1. SEQ ID No. 3, SEQ ID No. 5, SEQ ID No. 7 and SEQ ID No. 9; and the second Bremia-resistance providing genomic fragment is located on chromosome 8 of the lettuce plants, and comprises a nucleic acid sequence selected from the group consisting of SEQ ID No. 11, SEQ ID No. 13, SEQ ID No. 15, SEQ ID No. 17 and SEQ ID No. 19.

Alternatively, the above object, amongst other objects, is met by providing lettuce plants resistant to Bremia comprising a third and a fourth Bremia-resistance providing genomic fragment from Lactuca serriola, wherein the third Bremia-resistance providing genomic fragment is located on chromosome 4 of the lettuce plants and comprises a nucleic acid sequence selected from the group consisting of SEQ ID No. 31, SEQ ID No. 33, SEQ ID No. 35, SEQ ID No. 37 and SEQ ID No. 39; and the fourth Bremia-resistance providing genomic fragment is located on chromosome 8 of the lettuce plants, and comprises a nucleic acid sequence selected from the group consisting of SEQ ID No. 41. SEQ ID No. 43, SEQ ID No. 45, SEQ ID No. 47 and SEQ ID No. 49.

According to yet another alternative, the above object, amongst other objects is met by providing lettuce plants resistant to Bremia comprising a fifth Bremia-resistance providing genomic fragment from Lactuca serriola, wherein the fifth Bremia resistance providing fragment is located on chromosome 2 of the lettuce plants and comprises a sequence selected from the group consisting of SEQ ID No. 21, SEQ ID No. 23, SEQ ID No. 25, SEQ ID No. 27 and SEQ ID No. 29.

According to still another alternative, the above object, amongst other objects is met by providing lettuce plants resistant to Bremia, wherein the lettuce plants comprise a fifth Bremia-resistance providing genomic fragment from Lactuca serriola as defined above in combination with:
a) a first and a second Bremia-resistance providing genomic fragments from Lactuca serriola as defined above; or
b) a third and a fourth Bremia-resistance providing genomic fragments from Lactuca serriola as defined above.

### DESCRIPTION OF THE INVENTION

The present inventors have surprisingly identified new genetically encoded resistances against Bremia lactucae. The present genomic fragments conferring resistance to Bremia from L. serriola are different from the Bremia resistances described in WO2009111627, WO2011003783, WO2015136085 and EP0629343 that were introduced from L. saligna or WO2000063432 which originates from L. virosa.

Molecular markers are used as a tool by researchers aiming to introduce desired genomic regions comprising one or more resistance genes into cultivars. For example the presence or absence of the genes providing resistance to plant pathogens such as Bremia lactucae can be detected with molecular markers. This technique allows (breeders) to save time as compared to often laborious phytopathological tests. Testing with makers can be performed at all stages of plant development, which is not always the case for a reliable disease test. Moreover, the genetic tests for the presence of markers are not dependent on field circumstances which can or cannot promote the development of the disease and are generally less susceptible to experimental variation as compared to pathological test making them more reliable.

It can take considerable effort and a long time to develop informative markers. The nature of the resistance plays a role and the researchers need to find out whether the resistance is monogenic or multigenic and dominant or recessive. The availability and quality of the disease tests and the availability and the size of the test populations both have an effect on how difficult it is to develop the markers. Another crucial factor that has influence on the development of markers is the quality of the reference genome or the genetic map of the plants of interest.

Complex genomes can be challenging in the identification of informative markers like Single Nucleotide Polymorphism (SNPs) markers, which is partially caused by the presence of highly repetitive sequences in plant genomes. Markers used in plant breeding should be tightly linked to target loci, preferably less than 5 cM genetic distance, even more preferably flanking the gene of interest and ideally the markers being intragenic. However it remains laborious and time-consuming to define a haplotype block.

The progress in molecular genetics such as introduction of SNP markers, better (cheaper, easier accessible, faster) genome sequencing and increased availability and coverage of genomic data has led to development of more informative markers. It has also set the quality threshold higher for plant scientist developing markers. For example the AFLP or RAMP makers that were common practice 15 years ago would currently not be satisfactory for plant breeding companies. It therefore is anything but obvious to develop new markers and each new genetically encoded resistance needs a tailored approach.

According to a preferred embodiment, the present invention relates to a lettuce plant resistant to Bremia, wherein the Bremia-resistance provides resistance against races B1:33 to B1:36 and B1:25 to B1:31, preferably B1:16 to B1:37.

According to other preferred embodiments, the present first and the second Bremia-resistance providing genomic fragments are obtainable, obtained or are from a lettuce plant, the seeds of which are deposited under deposit number NCIMB 43820; the present third and the fourth Bremia-resistance providing genomic fragments are obtainable, obtained or are from a lettuce plant, the seeds of which are deposited under deposit number NCIMB 43819 or NCIMB 43818; and/or the present fifth Bremia-resistance providing genomic fragment is obtainable, obtained or is from a lettuce plant the seeds of which are deposited under deposit number NCIMB 43821 or NCIMB 43818.

The above deposits were deposited at NCIMB Limited, Craibstone Estate, Ferguson Building, Bucksburn, Aberdeen AB21 9YA, United Kingdom on Jul. 23, 2021.

According to a second aspect, the present invention relates to seeds, or progeny, of the present lettuce plants. Specifically, the present invention relates to seeds or progeny of the present lettuce plants resistant to Bremia, the seeds, or progeny, comprise in their genome:
a) the present first and second Bremia-resistance providing genomic fragments from Lactuca serriola, or
b) the present third and the fourth Bremia-resistance providing genomic fragments from Lactuca serriola, or
c) the present fifth Bremia-resistance providing genomic fragment from Lactuca serriola, or
d) the present fifth Bremia-resistance providing genomic fragment from Lactuca serriola in combination with:
   1) the present first and a second Bremia-resistance providing genomic fragments from Lactuca serriola, or
   2) the present third and a fourth Bremia-resistance providing genomic fragments from Lactuca serriola.

According to a third aspect, the present invention relates to edible parts, pollen, egg cells, callus, suspension culture, somatic embryos, clones, embryos or plant parts of the present lettuce plants resistant to Bremia.

The present invention also relates to methods for identifying Bremia resistant plants, the methods comprise the steps of:
i) isolating or providing the genomic nucleic acid of the plant,
ii) detecting in the isolated nucleic genomic acid:
   a) a first and said second Bremia-resistance providing genomic fragments from Lactuca serriola as defined above, and/or
   b) a third and a fourth Bremia-resistance providing genomic fragments from Lactuca serriola as defined above, and/or
   c) a fifth Bremia-resistance providing genomic fragment from Lactuca serriola as defined above.

According to a preferred embodiment, the present methods comprise a step (i) or step (ii) comprising nucleic acid amplification.

Considering the beneficial properties of the present Bremia-resistance providing genomic fragment from Lactuca serriola, the present invention also relates to the use of one or more of genomic sequences comprising one or more DNA sequences selected from the group consisting of: SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No. 27, SEQ ID No. 29, SEQ ID No. 31, SEQ ID No. 33, and SEQ ID No. 35, SEQ ID No. 37, SEQ ID No. 39, SEQ ID No. 41, SEQ ID No. 43, SEQ ID No. 45, SEQ ID No. 47 and SEQ ID No. 49, for identifying, or providing, a lettuce plant being resistant to Bremia.

Additionally, the present invention relates to use of one or more markers selected from the group consisting of: SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No. 27, SEQ ID No. 29, SEQ ID No. 31, SEQ ID No. 33, and SEQ ID No. 35, SEQ ID No. 37, SEQ ID No. 39, SEQ ID No. 41, SEQ ID No. 43, SEQ ID No. 45, SEQ ID No. 47 and SEQ ID No. 49 for identifying, or providing, a lettuce plant being resistant to Bremia.

The present Bremia-resistance providing genomic fragment, from Lactuca serriola, according to the present invention, can be used in methods for providing Bremia resistant lettuce plants, wherein the methods comprise the step of introgressing:
a) the present first and second Bremia-resistance providing genomic fragments from Lactuca serriola, or
b) the present third and the fourth Bremia-resistance providing genomic fragments from Lactuca serriola, or
c) the present fifth Bremia-resistance providing genomic fragment from Lactuca serriola, or
d) the present fifth Bremia-resistance providing genomic fragment from Lactuca serriola in combination with:
   1) the present first and a second Bremia-resistance providing genomic fragments from Lactuca serriola, or
   2) the present third and a fourth Bremia-resistance providing genomic fragments from Lactuca serriola.
      into a lettuce plant not comprising the first, the second, the third, the fourth and/or the fifth Bremia resistance providing genomic fragment.

Alternatively, the present Bremia-resistance providing genomic fragments from Lactuca serriola, according to the present invention, can be used in methods for providing Bremia resistant lettuce plants, wherein the Bremia resistance-providing genomic fragment or fragments from Lactuca serriola are introduced into a lettuce plant, wherein in the Bremia resistant lettuce plant is not exclusively obtained by means of an essentially biological process.

Not exclusively obtained by means of an essentially biological process can comprise at least the step of co-transformation with Agrobacterium and/or CRISPR/CAS.

For example, the resistances according to the present invention can be introduced in a lettuce cell by transformation (e.g., using Agrobacterium tumefaciens). Genomic fragments can be amplified by long-range PCR amplifications, de novo synthesized, or isolated from gels or columns (e.g., after restriction digestion). The resulting fragments can be reassembled (e.g., into yeast) or introduced in an expression vector, subsequently transformed into lettuce and allowed to integrate or recombine with the lettuce genome. The fragment may be introduced in a single step or in a series of transformations resulting in a lettuce plant comprising the resistance of the present invention GMO.

Alternatively, Tobacco rattle virus (TRV)-derived VIGS vectors can be employed. To confirm that the genomic fragments from L. serriola, namely: the genetic fragments encoding the resistance from L. serriola referred to as 140: Ch4+Ch8, the genetic fragments encoding the resistance from L. serriola referred to as 139: Ch4+Ch8 and or the genetic fragment encoding the resistance from L. serriola referred to as 139: Ch2; where these fragments are present in lettuce plants according to the invention, are responsible for the observed resistance phenotype, a VIGS experiment can be carried out.

For this purpose VIGS constructs, targeting the sequences encoded by said genomic fragments, can be designed and synthesized or otherwise obtained with molecular biology techniques and cloned into a VIGS vector. The said vectors can be subsequently transformed into L. sativa plants according to this invention using co-cultivation with Agrobacterium tumefaciens.

The present invention furthermore relates to nucleic acid sequence selected from the group consisting of: SEQ ID No. 1, SEQ ID No. 3, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, SEQ ID No. 23, SEQ ID No. 25, SEQ ID No. 27, SEQ ID No. 29, SEQ ID No. 31, SEQ ID No. 33, and SEQ ID No. 35, SEQ ID No. 37, SEQ ID No. 39, SEQ ID No. 41, SEQ ID No. 43, SEQ ID No. 45, SEQ ID No. 47, and SEQ ID No. 49.

The present invention will be further detailed in the examples below.

### EXAMPLES

### Example 1. Lettuce Plants Comprising Bremia-Resistance Providing Genomic Fragments from Lactuca serriola and Their Deposits

Representative seeds described below were deposited at the NCIMB, NCIMB Limited, Ferguson Building; Craibstone Estate, Bucksburn ABERDEEN, Scotland, AB21 9YA, United Kingdom) on 23 Jul. 2021 under deposit numbers: NCIMB 43818, NCIMB 43819, NCIMB 43820 and NCIMB 43821.

A first plant lettuce designated “140” comprises Bremia-resistance providing genomic fragments from Lactuca serriola, wherein a first Bremia resistance providing genomic fragment is located on chromosome 4 and a second Bremia resistance providing genomic fragment is located on chromosome 8. A representative seed lot was deposited under deposit number NCIMB 43820.

A second lettuce plant designated “139” comprises Bremia-resistance providing genomic fragments from Lactuca serriola, wherein a third Bremia resistance providing genomic fragment is located on chromosome 4 and a fourth Bremia resistance providing genomic fragment is located on chromosome 8 and a fifth Bremia resistance providing genomic fragment is located on chromosome 2. A representative seed lot was deposited under deposit number NCIMB 43818. In plant “139”, the fifth Bremia-resistance providing genomic fragment from Lactuca serriola located on chromosome 2 is and provides resistance to Bremia independently from the other fragments therein.

A representative seed lot of lettuce plants comprising a fifth Bremia-resistance providing genomic fragment from Lactuca serriola wherein the Bremia resistance providing fragment is located on chromosome 2 was deposited under deposit number NCIMB 43821.

In the lettuce plants designated “139”, the Bremia-resistance providing genomic fragments from Lactuca serriola, wherein the third Bremia resistance providing genomic fragment is located on chromosome 4 and the fourth Bremia-resistance providing genomic fragment from Lactuca serriola is located on chromosome 8, provide resistance against Bremia independently from the fifth Bremia-resistance providing genomic fragment from Lactuca serriola located on chromosome 2. A representative seed lot of these plants has been deposited under deposit number NCIMB 43819.

### Example 2. Disease Test

The Bremia disease tests are performed in a climate chamber at 18° C./15° C. and 70%/100% RH during 16/8 hours day/night periods, respectively. Bremia spores are harvested from seedlings of a susceptible variety by suspending spores in water. The Bremia spore suspension is then used to inoculate the Bremia disease test. The seedlings are assessed 10 days after inoculation and scored + if sporulation is observed, − if sporulation is absent, (−) if necrosis is observed and (+) if reduced sporulation is observed.

The choice of a susceptible variety for a Bremia isolate is made from the official differential host set (Table 1). Disease testing for Bremia resistance was performed with Bremia races that are officially described by the IBEB-EU (B1: 16EU to B1 : 37EU). Confirmation of the used race in the disease test was done by inoculating the official differential host set (Table 1). Performance of seeds of which representative samples have been deposited (see example 1) was compared to currently known Lactuca varieties.

TABLE 1
The official international differential host set of the IBEB-EU for identification of Bremia races in lettuce
Bl: 16EU-Bl: 37EU. The symbols indicate + sporulation, − no sporulation, (−) necrosis, (+) reduced sporulation.

| | | | | UC | | | | FrRsal- | | RYZ | |
|---|---|---|---|---|---|---|---|---|---|---|---|
| | GreenTowers | Dandie | R4T57D | Dm14 | NunDm15 | CGDm16 | Colorado | 1 | Argelés | 2164 | RYZ910457 |
| Sextet value | | 1 | 2 | 4 | 8 | 16 | 32 | 1 | 2 | 4 | 8 |
| Bl: 16EU | + | + | + | − | − | + | − | − | − | − | − |
| Bl: 17EU | + | + | − | + | + | − | + | + | − | − | − |
| Bl: 18EU | + | − | + | − | − | + | + | − | − | − | − |
| Bl: 20EU | + | + | + | − | − | + | + | − | − | − | − |
| Bl: 21EU | + | + | + | − | + | + | − | + | − | − | − |
| Bl: 22EU | + | − | + | + | + | − | + | − | − | − | − |
| Bl: 23EU | + | + | + | − | − | + | − | − | + | − | − |
| Bl: 24EU | + | − | + | − | − | + | + | − | + | − | − |
| Bl: 25EU | + | − | + | − | − | + | + | + | − | − | − |
| Bl: 26EU | + | + | + | − | − | + | + | + | + | − | − |
| Bl: 27EU | + | + | + | + | + | − | + | − | + | + | − |
| Bl: 29EU | + | − | + | + | + | + | + | + | + | + | − |
| Bl: 30EU | + | − | + | + | + | − | + | − | + | + | − |
| Bl: 31EU | + | + | + | + | − | − | + | − | − | + | + |
| Bl: 32EU | + | + | + | − | + | + | − | − | − | − | − |
| Bl: 33EU | + | − | + | + | + | + | + | + | + | + | − |
| Bl: 34EU | + | − | + | + | − | + | + | + | + | + | + |
| Bl: 35EU | + | − | + | + | + | + | + | + | + | + | + |
| Bl: 36EU | + | + | + | + | − | + | + | + | + | + | + |
| Bl: 37EU | + | − | + | + | + | − | + | + | + | + | + |

| | | | | | | | | | | | D |
|---|---|---|---|---|---|---|---|---|---|---|---|
| | GreenTowers | Bedford | Balesta | Bartoli | Design | Kibrille | Fenston | Bataille | RYZ20007 | | sextet |
| Sextet value | | 16 | 32 | 1 | 2 | 4 | 8 | 16 | 32 | Set | code |
| Bl: 16EU | + | − | − | − | − | − | − | − | − | D | 19-00-00 |
| Bl: 17EU | + | (+) | − | − | − | − | − | − | − | D | 45-17-00 |
| Bl: 18EU | + | − | − | − | − | − | − | − | − | D | 50-00-00 |
| Bl: 20EU | + | − | − | − | − | − | − | − | − | D | 51-00-00 |
| Bl: 21EU | + | − | − | − | − | − | − | − | − | D | 27-01-00 |
| Bl: 22EU | + | − | + | − | − | − | − | − | − | D | 46-32-00 |
| Bl: 23EU | + | − | − | − | − | − | − | − | − | D | 19-02-00 |
| Bl: 24EU | + | − | − | − | (−) | − | (−) | (−) | − | D | 50-02-00 |
| Bl: 25EU | + | − | − | − | − | − | − | − | − | D | 50-01-00 |
| Bl: 26EU | + | − | − | − | − | − | − | − | − | D | 51-03-00 |
| Bl: 27EU | + | − | + | − | − | − | − | − | − | D | 47-38-00 |
| Bl: 29EU | + | − | − | − | − | − | − | − | − | D | 62-07-00 |
| Bl: 30EU | + | − | − | − | + | − | − | − | − | D | 46-06-02 |
| Bl: 31EU | + | − | − | − | + | − | − | − | − | D | 39-12-02 |
| Bl: 32EU | + | − | − | − | − | + | − | − | − | D | 27-00-04 |
| Bl: 33EU | + | − | − | − | + | + | − | − | − | D | 62-07-06 |
| Bl: 34EU | + | − | − | + | (−) | − | − | − | − | D | 54-15-01 |
| Bl: 35EU | + | − | − | − | + | + | − | − | − | D | 62-15-06 |
| Bl: 36EU | + | − | − | + | − | − | − | − | − | D | 55-15-01 |
| Bl: 37EU | + | − | − | − | + | + | + | − | − | D | 46-15-14 |

TABLE 2
Resistance pattern of plants according to the invention against
Bremia races in lettuce Bl: 16EU-Bl: 37EU. The symbols
indicate +sporulation, −no sporulation, (−) necrosis,
(+) reduced sporulation, n.t.-not tested.

| | 140:C4 + C8 | 139:C2 + C4 + C8 | 139:C2 | 139:C4 + C8 |
|---|---|---|---|---|
| Bl: 16EU | − | − | − | − |
| BI: 17EU | − | − | − | − |
| Bl: 18EU | − | − | − | − |
| Bl: 20EU | − | − | − | − |
| Bl: 21EU | − | − | n.t. | n.t. |
| Bl: 22EU | − | − | − | − |
| Bl: 23EU | − | − | − | − |
| Bl: 24EU | − | − | n.t. | n.t. |
| Bl: 25EU | − | − | − | − |
| Bl: 26EU | − | − | − | − |
| Bl: 27EU | − | − | − | − |
| Bl: 29EU | − | − | − | − |
| Bl: 30EU | − | − | − | − |
| Bl: 31EU | − | − | − | − |
| Bl: 32EU | − | − | n.t. | − |
| Bl: 33EU | − | − | − | − |
| Bl: 34EU | − | − | − | − |
| Bl: 35EU | − | − | − | − |
| Bl: 36EU | − | − | − | − |
| Bl: 37EU | − | − | n.t. | n.t. |

### Example 3. Development of Markers for Plant/Resistance Named 140 with Resistance on Chromosomes 4 and 8 (140: C4+C8)

Genotypes were screened for resistance against BI: 16-36 and a L. serriola was found to be resistant against all races. A segregating F1S1 population was developed to map the resistance. A population derived from the Bremia resistant L. serriola and a susceptible L. sativa was analyzed to identify the genomic location(s) of the resistance.

The F1S1 population consisted of 474 seedlings. From those 474 seedlings, 237 were susceptible for B. lactucae isolates, 44 showed an intermediate phenotype and 193 showed no disease symptoms. Informative genome-wide markers were developed to genotype the mapping population. The genotype was determined for 363 F1S1 plants with 196 informative markers. The used reference genome was V8 (Reyes-Chin-Wo et al., 2017).

All genotyped susceptible plants had an allele from the susceptible parent on chromosome 4 between 282.9 and 291.3 Mbp or chromosome 8 between 47.5 and 56.8 Mbp. All genotyped resistant plants had at least one allele from the resistant parent on both loci. Most genotyped plants that showed an intermediate phenotype were heterozygous at the locus on chromosome 4.

To make sure the locus on chromosome 4 and chromosome 8, without any other locus, provided resistance against at least B1:16-36, the loci were introgressed into a susceptible background. This new line was tested on Bremia races B1:16-36.

It is concluded that the resistance derived from the aforementioned L. serriola is a polygenic resistance which localizes on chromosome 4 between 282.9 and 291.3.8 Mbp and chromosome 8 between 47.5 and 56.8 Mbp in L. sativa. The offspring of plants, homozygous for the allele that confers resistance on chromosome 4 between 282.9 and 291.3.8 Mbp and chromosome 8 between 47.5 and 56.8 Mbp, showed full resistance against twenty Bremia isolates.

A lettuce line containing the resistance was sequenced and additional SNP markers were developed within the intogression. The newly developed markers were to fine-map the locus on chromosome 4 to a region between 284.4 Mbp and 286.7 Mbp and the locus on chromosome 8 to a region between 47.5 Mbp and 56.1 Mbp. The newly developed markers within the fine-mapped loci co-segregated with the resistance in all breeding material tested.

TABLE 3
SNPs for the detection of the resistance against Bremia lactucae.

| | | Position | Allele linked | Alternative |
|---|---|---|---|---|
| SNP | Chromosome | Chromosome* (bp) | to resistance | allele |
| 1 | chr4 | 284.651.859 | A | C |
| 2 | chr4 | 285.408.314 | T | C |
| 3 | chr4 | 286.308.410 | A | C |
| 4 | chr4 | 286.429.405 | C | T |
| 5 | chr4 | 286.693.874 | C | G |
| 6 | chr8 | 47.911.009 | C | A |
| 7 | chr8 | 49.507.124 | C | G |
| 8 | chr8 | 50.016.400 | T | G |
| 9 | chr8 | 51.748.617 | C | A |
| 10 | chr8 | 52.830.615 | T | C |

The reference genome is: V8 (Reyes-Chin-Wo et al., 2017)

TABLE 4
Sequences of the SNPs for the detection
of the resistance against Bremia lactucae.
Odd sequences = linked to resistance,
Even sequences = alternative allele (L. sativa cv Salinas).

| SEQ | | | Position | |
|---|---|---|---|---|
| ID | | | Chromosome* | |
| no. | SNP | Chromosome* | (bp) | Sequence |
| SEQ | 1 | 4 | 284.651.859 | ATTTTGATTGGTTGATAAGAATTTCACTACAA |
| ID | | | | CACCAAATGATTCTCGAC[A]TCTAGCAGTGGT |
| No. 1 | | | | AGAATTTCTCGAACTCATTTTTATCGAGTTTTA |
| | | | | ACAAA |
| SEQ | 1 | 4 | 284.651.859 | ATTTTGATTGGTTGATAAGAATTTCACTACAA |
| ID | | | | CACCAAATGATTCTCGAC[C]TCTAGCAGTGGT |
| No. 2 | | | | AGAATTTCTCGAACTCATTTTTATAGAGTTTTA |
| | | | | ACAAA |
| SEQ | 2 | 4 | 285.408.314 | CTGATTTGAGATCTTGCACACTCCTAATAATA |
| ID | | | | ACACAATACATGGTCTAA[T]TTTGTTTTTGTTT |
| No. 3 | | | | ATTTTGCTTCTCTTGGTACCATGTTCGTATGCA |
| | | | | CACA |
| SEQ | 2 | 4 | 285.408.314 | CTGATTTGAGACCTTGCACACTCCTAATAATA |
| ID | | | | ACACAATACATGGTCTAA[C]TTTGTTTTTGTTT |
| No. 4 | | | | ATTTTGCTTCTCTTGGTACCATGTTCGTATGCA |
| | | | | CACA |
| SEQ | 3 | 4 | 286.308.410 | CAAAAATGGCCGCCAGAATAGCTCGTAACTG |
| ID | | | | CAACACACCAAAGAGTCTC[A]CAATCGAATG |
| No. 5 | | | | ACACGTAGAAAACAATTGTTTACCGCAGTCAA |
| | | | | AATTTCGC |
| SEQ | 3 | 4 | 286.308.410 | CAAAAATGGCCGCCAGAATAGCTCGTAACTG |
| ID | | | | CAACACACCAAAGAGTCTC[C]CAATCGAATG |
| No. 6 | | | | ACACGTAGAAAACAATTGTTTACCGCAGTCAA |
| | | | | ATTTTCGC |
| SEQ | 4 | 4 | 286.429.405 | GCAAAGAAGGGTCCTGCTAAGGAAACATGAG |
| ID | | | | GATGAGAAGGGCATTTATG[C]CTTTTTCTCAC |
| No. 7 | | | | CCGGTTTTTTATTGTTGTTTTCTTATTTGACCTT |
| | | | | TTGGA |
| SEQ | 4 | 4 | 286.429.405 | GCAAAGAAGGGTCCTGCTAAGGAAACATGAG |
| ID | | | | GATGAGAAGGGCATTTATG[T]CTTTTTCTCAC |
| No. 8 | | | | CCGGTTTTTTATTGTTGTTTTCTTATTTGACCTT |
| | | | | TTGGA |
| SEQ | 5 | 4 | 286.693.874 | ACACAACCAAATAATAAAATAAAATAATTTTC |
| ID | | | | ATAAATTATACTTAGAGG[C]TGTTTGGATGTA |
| No. 9 | | | | CTTTTTAATCTCAAAAGTGTTTTTGGGAAAAG |
| | | | | TCTTTA |
| SEQ | 5 | 4 | 286.693.874 | ACACAACCAAATAATAAAATAAAATAATTTTC |
| ID | | | | ATAAATTATACTTAGAGG[G]TGTTTGGATGTA |
| No. 10 | | | | CTTTTTAATCTCAAAAGTGTTTTTGGGAAAAG |
| | | | | TCTTTA |
| SEQ | 6 | 8 | 47.911.009 | GGAAAGATTTGTGTGCTTTGATTCCATTTGCA |
| ID | | | | GGTATAGTTTTATTTCAA[C]ATTTTGGTTCATG |
| No. 11 | | | | ATGTTTCAAATGATGGTTGGAACACATGAATT |
| | | | | TGATT |
| SEQ | 6 | 8 | 47.911.009 | GGAAAGATTTGTGTGCTTTGATTCCATTTGCA |
| ID | | | | GGTATAGTTTTATTTCAA[A]ATTTTGGTTCATG |
| No. 12 | | | | ATGTTTCAAATGATGTTTGGAACACATGAATT |
| | | | | TGATT |
| SEQ | 7 | 8 | 49.507.124 | TGGATCCTTGTGGTCATTTTGTGTTATATGTTT |
| ID | | | | AAGATCTAAGGATCCTT[C]GAGTTTTGTTCCC |
| No. 13 | | | | ATGCTCAAGCTTTAAGTACTCATTTGCCACTT |
| | | | | CTCAT |
| SEQ | 7 | 8 | 49.507.124 | TGGATCCTTGTGGTCATTTTGTGTTATATGTTT |
| ID | | | | AAAATCTAAGGATCCTT[G]GAGTTTTGTTCCC |
| No. 14 | | | | ATGCTCAAGCTTTAAGTACTCATTTGCCACTT |
| | | | | CTCAT |
| SEQ | 8 | 8 | 50.016.400 | AAACAACAAGAATTGGTGGTTTACTCACCTCC |
| ID | | | | CAAGTGGAATTAATTAAT[T]GGTTCAAGCAGA |
| No. 15 | | | | AAAGCTCAAAACCCCAGTAATTAAATTAAAT |
| | | | | AACCTAA |
| SEQ | 8 | 8 | 50.016.400 | AAACAACAAGAATTGGTGGTTTACTCACCTCC |
| ID | | | | CAAGTGGAATTAATTAAT[G]GGTTCAAGCAG |
| No. 16 | | | | AAAAGCTCAAAACCCCAGTAATTAAATTAAA |
| | | | | TAACCTAA |
| SEQ | 9 | 8 | 51.748.617 | ATTACAAGAGGATTAATGCATATAAGTTTAGA |
| ID | | | | ATCTAACAGATTTCAGTA[C]ATGGCATGTAAG |
| No. 17 | | | | TGCCTAAATTCGCCTGTGTATCAATCAGATCC |
| | | | | ATTTGA |
| SEQ | 9 | 8 | 51.748.617 | ATTACAAGAGGATTAATGCATATAAGTTTAGA |
| ID | | | | ATCTAACAGATTTCAGTA[A]ATGGCATGTAAG |
| No. 18 | | | | TGCCTAAATTCGCCTGTGTATCAATCAGATCC |
| | | | | ATTTGA |
| SEQ | 10 | 8 | 52.830.615 | AGTTAGAGTTTCATACCATGCATGAGATGCTT |
| ID | | | | GTTTTAAGCCGTACACCG[T]CTTATCCAAAAT |
| No. 19 | | | | TATAACATGGTTTGGAAATTCCTCGTTTACGA |
| | | | | AACCTG |
| SEQ | 10 | 8 | 52.830.615 | AGTTAGAGTTTCATACCATGCATGAGATGCTT |
| ID | | | | GTTTTAAGCCGTACACCG[C]CTTATCCAAAAG |
| No. 20 | | | | TATAACATGGTTTGGAAATTCCTCGTTTACGA |
| | | | | AACCTG |

*The reference genome is: V8 (Reyes-Chin-Wo et al., 2017)

### Nucleotide Base Codes According to the International Union of Pure and Applied Chemistry (IUPAC) Code:

| Symbol | Nucleotide Base |
|---|---|
| A | Adenine |
| C | Cytosine |
| G | Guanine |
| T | Thymine |
| N | A or C or G or T |
| M | A or C |
| R | A or G |
| W | A or T |
| S | Cor G |
| Y | C or T |
| K | G or T |
| V | Not T |
| H | Not G |
| D | Not C |
| B | Not A |

### Example 4. Development of Markers for Plant/Resistance Named 139 with Resistance on Chromosomes 2, 4 and 8 (139: C2 and 139: C4+C8)

Many genotypes were screened for resistance against BI: 16-36 and a L. serriola was found to be resistant against all races. A segregating F1S1 population was developed to map the resistance. A population derived from the Bremia resistant L. serriola and a susceptible L. sativa was analyzed to identify the genomic location(s) of the resistance.

The F1S1 population consisted of 476 seedlings. After inoculation with B. lactucae, 50 showed clear sporulation, 131 showed an intermediate phenotype and 295 did not show any disease symptoms. Informative genome-wide markers had to be developed to genotype the mapping population. The genotype was determined for 476 F1S1 plants with 196 informative markers. The used reference genome was V8 (Reyes-Chin-Wo et al., 2017).

Almost all susceptible plants (49 out of 50) had an allele from the susceptible parent on chromosome 4 between 283.4 and 286.9 Mbp and/or chromosome 8 between 49.5 and 56.8 Mbp. Most resistant plants had at least one allele from the resistant parent at these loci. Moreover, there were no susceptible plants that were homozygous for the resistant parent at chromosome 2 between 6.3 and 11.8 Mbp.

To study the loci on chromosome 4 and chromosome 8, both loci were introgressed into a susceptible background. The newly developed line, containing both loci, was tested on Bremia races B1:33-36. The locus on chromosome 2 was not present in the new line.

It is concluded that the aforementioned L. serriola contains a polygenic resistance which localizes on chromosome 4 between 284.4 and 286.8 Mbp and on chromosome 8 between 47.5 and 56.1 Mbp in L. sativa. L. sativa plants containing a homozygous introgression from the aforementioned L. serriola on chromosome 4 between 284.4 and 286.8 Mbp and on chromosome 8 between 47.5 and 56.1 Mbp, showed full resistance against four Bremia isolates.

To make sure the locus on chromosome 2, without any other locus, provided resistance at least against B1:33-36, the locus was introgressed into a susceptible background. The loci on chromosome 4 and chromosome 8 were not present in this new line. The new line was tested with Bremia races B1:33-36.

It is concluded that part of the resistance derived from the aforementioned L. serriola is a monogenic resistance which localizes on chromosome 2 between 8.6 and 10.6 Mbp in L. sativa. The offspring of plants, homozygous for the allele on chromosome 2 between 8.6 and 10.6 Mbp that confers resistance, showed full resistance against four Bremia isolates.

A lettuce line containing the resistance was sequenced and additional SNP markers were developed within the introgressions. The newly developed markers within the fine-mapped loci co-segregated with the resistance in all breeding material tested.

TABLE 5
SNPs for the detection of the resistance against Bremia lactucae.

| | | Position | Allele linked | Alternative |
|---|---|---|---|---|
| SNP | Chromosome | Chromosome* (bp) | to resistance | allele |
| 11 | chr2 | 8.601.189 | A | G |
| 12 | chr2 | 9.082.766 | A | C |
| 13 | chr2 | 9.161.964 | G | A |
| 14 | chr2 | 10.168.534 | T | A |
| 15 | chr2 | 10.211.004 | T | G |
| 16 | chr4 | 284.399.793 | C | T |
| 17 | chr4 | 284.674.740 | G | T |
| 18 | chr4 | 285.180.325 | G | C |
| 19 | chr4 | 286.072.468 | G | A |
| 20 | chr4 | 286.801.599 | G | A |
| 21 | chr8 | 49.539.660 | G | A |
| 22 | chr8 | 50.797.252 | A | G |
| 23 | chr8 | 51.494.054 | A | C |
| 24 | chr8 | 52.177.187 | G | A |
| 25 | chr8 | 53.782.739 | T | C |

*The reference genome is: V8 (Reyes-Chin-Wo et al., 2017)

TABLE 6
Sequences of the SNPs for the detection of the resistance
against Bremia lactucae in the plant/resistance named 139.
Odd sequences = linked to resistance,
Even sequences = alternative allele (L. sativa cv Salinas).

| SEQ | | | Position | |
|---|---|---|---|---|
| ID | | | Chromosome* | |
| no. | SNP | Chromosome* | (bp) | Sequence |
| SEQ | 11 | 2 | 8.601.189 | AACTGCTTGTTTTTATATATTTCCCTCTTGC |
| ID | | | | AGATGTTTTACATGGAAGA[A]TGAGAGGTT |
| No. | | | | GCTTGTGGAAAGCCACCGATTACATGTCAA |
| 21 | | | | ATAAAGTCTTT |
| SEQ | 11 | 2 | 8.601.189 | AACTGCTTGTTTTTATATATTTCCCTCTTGC |
| ID | | | | AGATCTTTTACATGGAAGA[G]TGAGAGGTT |
| No. | | | | GCTTGTGGAAAGCCACCGATTACATGTCAA |
| 22 | | | | ATAAAGTCTTT |
| SEQ | 12 | 2 | 9.082.766 | AGCTCTTGGAGGAGCATCTCACGTCTTTTT |
| ID | | | | GAGCCAAGATTGTTGCGGGG[A]AGATTGG |
| No. | | | | GGTGTGAACTCCCTCATTTCGGGAGTTCAA |
| 23 | | | | GGTGTGTAGAGTT |
| SEQ | 12 | 2 | 9.082.766 | AGCTCTTGGAGGAGCATCTCACGTCTTTTT |
| ID | | | | GAGCCAAGATTGTTGCGGGG[C]AGATTGG |
| No. | | | | GGTGTGAACTCCCTCATTTCGGGAGTTCAA |
| 24 | | | | GGTGTGTAGAGTT |
| SEQ | 13 | 2 | 9.161.964 | AGAAAAACTCCCGCATCCTTGCACTTTGGA |
| ID | | | | GGTAAACCCTTTTCCAAAAT[G]TTTGAAAT |
| No. | | | | GTTTTCACTAGCTTTTAGTGTTTCGTTAGTT |
| 25 | | | | TTAATTTTTTT |
| SEQ | 13 | 2 | 9.161.964 | AGAAAAACTCCCGCATCCTTGCACTTTGGA |
| ID | | | | GGTAAACCCTTTTCCAAAAT[A]GTTGAAAT |
| No. | | | | GTTTTCACTAGCTTTTAGTGTTTCGTTAGTT |
| 26 | | | | TTAATTTTTTT |
| SEQ | 14 | 2 | 10.168.534 | TGGTTAATTTTTGAAAAGCGAAAGGCAGTT |
| ID | | | | AAGTGATTATAATTTTCTAA[T]AGATTGAT |
| No. | | | | CTTAACTTGTACAAACTTTCAAATAGGAAA |
| 27 | | | | ACCCATACTCTA |
| SEQ | 14 | 2 | 10.168.534 | TGGTTAATTTTTGAAAAGCGAAAGGCAGTT |
| ID | | | | AAGTGATTATAATTTTCTAA[A]AGATTGAT |
| No. | | | | CTTAACTTGTACAAACTTTCAAATAGGAAA |
| 28 | | | | ACCCATACTCTA |
| SEQ | 15 | 2 | 10.211.004 | TGGAAACTCAAAGACTGTCCATTGATTTCT |
| ID | | | | CTTCCATAGATTCCTTAAAC[T]ATCTAACCC |
| No. | | | | CACTAACTCCAGTTGTGTTAGGTTTGGAGG |
| 29 | | | | ATTGATAAGAG |
| SEQ | 15 | 2 | 10.211.004 | TGGAAACTCAAACACTGTCCATTGATTTCT |
| ID | | | | CTTCCATAGATTCCTTAAAC[G]ATCTAATC |
| No. | | | | CCACTAACTCCAGTTGTGTTAGGTTTGGAG |
| 30 | | | | GATTGATAAGAG |
| SEQ | 16 | 4 | 284.399.793 | TTTTAAATTTATGAAGGGCTTCATCCTTAG |
| ID | | | | AATGTAATAGATATACACAA[C]AAAATTAG |
| No. | | | | AATAATCATTTATAAAGATAACCATGTATT |
| 31 | | | | TCTTACTACTTA |
| SEQ | 16 | 4 | 284.399.793 | TTTTAAATTTATGAAGGGCTTCATCCTTAG |
| ID | | | | AATGTAATAGATATACACAA[T]AAAATTAG |
| No. | | | | AATAATCATTTATAAAGATAACCATGTATT |
| 32 | | | | TCTTACTACTTA |
| SEQ | 17 | 4 | 284.674.740 | CGAGACATTACATCGATTCAGGTGAGTTTC |
| ID | | | | GCCTCTCTGTACCCGTGGGT[G]GAAGGCAC |
| No. | | | | CAATGTCAGCCCACTAGTTTTGGTTATATG |
| 33 | | | | TTAGTAGAAGGG |
| SEQ | 17 | 4 | 284.674.740 | CGAGACATTACATCGATTCAGGTGAGTTTC |
| ID | | | | GCCTCTCTGTACCCGTGGGT[T]GAAGGCAC |
| No. | | | | CAATGTCAGCCCACTAGTTTTGGTTATATG |
| 34 | | | | TTAGTAGAAGGG |
| SEQ | 18 | 4 | 285.180.325 | AAAAGCATAGTATTGGAATGGTGGACATGT |
| ID | | | | CGGAATATTATGTTACCCCA[G]ACAAGTGT |
| No. | | | | AGGACCAATAAGACTAATCAACATCATTTT |
| 35 | | | | GAAGTTGTGATT |
| SEQ | 18 | 4 | 285.180.325 | AAAAGCATAGTATTGGAATGGTGGACATGT |
| ID | | | | CGGAATATTATGTTACCCCA[C]ACAAGTGT |
| No. | | | | AGGACCAATAAGACTAATCAACATCATTTT |
| 36 | | | | GAAGTTGTGATT |
| SEQ | 19 | 4 | 286.072.468 | CTGTTCATTTTTTGAATTTCGTCTGAAGTTT |
| ID | | | | TTGACGATGAAGAACGAAG[G]ATCTATCTA |
| No. | | | | TCTGTTCTAATGTTGTTTGTGTTTAGGTGAA |
| 37 | | | | CAACAAACCC |
| SEQ | 19 | 4 | 286.072.468 | ATGTTCATTTTTTGAATTTCGTCTGAAGTTT |
| ID | | | | TTGACGATGAAGAACGAAG[A]ATCTATCTA |
| No. | | | | TCTGTTCTAATGTTGTTTGTGTTTAGGTGAA |
| 38 | | | | CAATAAACCC |
| SEQ | 20 | 4 | 286.801.599 | AAGGCTGGAGGGGCATACACAACTATAAA |
| ID | | | | AAGCACTTCTAGGCCCGAACG[G]ATCCAAA |
| No. | | | | CCGCCTGAATAGCTACATACCAATCATGAA |
| 39 | | | | TCGAAACCCGTGT |
| SEQ | 20 | 4 | 286.801.599 | AAGGCTTGAGGGGCATACACAACTATAAA |
| ID | | | | AAGCACTTCTAGGCCCGAACG[A]ATCCAAA |
| No. | | | | CCGCCTGAATTGCTACATACCAATCATGAA |
| 40 | | | | TCGAAACCCGTGT |
| SEQ | 21 | 8 | 49.539.660 | AGATTCACTAATCCATCAATGGAGAAACAC |
| ID | | | | CCAGAAAGATGAAGATCAAG[G]TGCTTAC |
| No. | | | | AACCCAATGACGAGTTTATCTGAAGAACAA |
| 41 | | | | GTGATCATCGTCC |
| SEQ | 21 | 8 | 49.539.660 | AGATTCACTAATCCATCAATGGAGAAACAC |
| ID | | | | CCAGAAAGATGAAGATCAAG[A]TGCTTAC |
| No. | | | | AACCCAATGACGAGTTTATCTGAAGAACAA |
| 42 | | | | GTGATCATCGTCC |
| SEQ | 22 | 8 | 50.797.252 | TTTCAGGTACTTCTGGCTCTAAGGGTAAAG |
| ID | | | | GCCCGACTTGATGGTGCATC[A]TACACTCT |
| No. | | | | CCAATATTCCGCACTAGTTGTTTTGGATACT |
| 43 | | | | TTGATCTTTTG |
| SEQ | 22 | 8 | 50.797.252 | TTTCAGGTACTTCTAGCTCTAAGGGTAAAG |
| ID | | | | GCCCGACTTGATGGTGCATC[G]TACACTCT |
| No. | | | | CCAATATTCCGCACTAGTTGTTTTGGATACT |
| 44 | | | | TTGATCTTTTG |
| SEQ | 23 | 8 | 51.494.054 | AAGATGCTTTCAAAGCACACACCACTCGAT |
| ID | | | | TCCGCATTTTGTGATTTACT[A]TGATATTGA |
| No. | | | | TGATGTATCTATACATTTTGATAACTTTGGT |
| 45 | | | | TTTATAAAAA |
| SEQ | 23 | 8 | 51.494.054 | AAGATGCTTTCAAAGCACACACCACTCGAT |
| ID | | | | TCCGCATTTTGTGATTTACT[C]TGATATTGA |
| No. | | | | TGATGTATCTATACATTTTGATAACTTTGGT |
| 46 | | | | TTTATAAAAA |
| SEQ | 24 | 8 | 52.177.187 | AACACCTCCAAGAGCCAAAAAGGACAAAT |
| ID | | | | CATTTGGACATAATCATCTAA[G]ACTCAAA |
| No. | | | | GGACCTCAAGTTGGGGACCAAAATGTGAC |
| 47 | | | | ATAAAACACCCAAA |
| SEQ | 24 | 8 | 52.177.187 | AACACCTCCAAGAGCCAAAAAGGACAAAT |
| ID | | | | CATTTGGACATAATCATCTAA[A]ACTCAAA |
| No. | | | | GGACCTCAAGTTGGGGACCAAAATGTGAC |
| 48 | | | | ATAAAACACCCAAA |
| SEQ | 25 | 8 | 53.782.739 | TATTAATCCATTAAGACATCACAAATCTTG |
| ID | | | | CATGGAAGGGATAGAAGGAA[T]GAGATCA |
| No. | | | | CATTTTTATGGTTCCTAATAGCTCCATAATG |
| 49 | | | | GATAAAGTTTCA |
| SEQ | 25 | 8 | 53.782.739 | TATTAATCCATTAAGACATCACAAATCTTG |
| ID | | | | CATGGAAGGGATAGAAGGAA[C]GAGATCA |
| No. | | | | CATTTTTATGGTTCCTAATAGCTCCATAATG |
| 50 | | | | GATAAAGTTTCA |

*The reference genome is: V8 (Reyes-Chin-Wo et al., 2017)

### Example 5. Introduction of the Genetic Fragments Providing Bremia Resistance From Lactuca serriolaInto Lactuca sativa With Agrobacterium

Transformation of plants with resistance genes using the Agrobacterium tumefaciens system can be a useful way of creating plants resistant to pathogens. To this end constructs harbouring the genetically encoded resistances according to this invention can be designed and synthesized or otherwise obtained with molecular biology techniques. These constructs would be harboring:
(1) The genetic fragments encoding the resistance from L. serriola referred to as 140: Ch4+Ch8;
(2) The genetic fragments encoding the resistance from L. serriola referred to as 139: Ch4+Ch8;
(3) The genetic fragment encoding the resistance from L. serriola referred to as 139: Ch2;

Susceptible L. sativa plants can be transformed with construct (1), construct (2), construct (3) or co-transformed with constructs (2) and (3) or (1) and (3) using co-cultivation with Agrobacterium. Upon completed transformation, stable transformants can be subjected to a disease test using a Bremia race B1:36. It is expected that the stable transformants will be resistant to infection with said Bremia race, while the non-transformed plants are susceptible.

### Example 6. Virus Induced Gene Silencing Experiment (VIGS) to Silence the Genetically Encoded Bremia Resistance

Tobacco rattle virus (TRV)-derived VIGS vectors have been abundantly described to study gene function in Arabidopsis thaliana, Nicotiana benthamiana, Lycopersicon esculentum and other plants.

To confirm that the genomic fragments from L. serriola, namely:
(1) The genetic fragments encoding the resistance from L. serriola referred to as 140: Ch4+Ch8;
(2) The genetic fragments encoding the resistance from L. serriola referred to as 139: Ch4+Ch8;
(3) The genetic fragment encoding the resistance from L. serriola referred to as 139: Ch2; where these fragments are present in L. sativa plants are responsible for the observed resistance phenotype, a VIGS experiment can be carried out.

For this purpose VIGS constructs, targeting the sequences encoded by said genomic fragments, can be designed and synthesized or otherwise obtained with molecular biology techniques and cloned into the VIGS vector. The said vectors can be subsequently transformed into L. sativa plants according to this invention using co-cultivation with Agrobacterium tumefaciens, as specified below:

| VIGS construct target | Target plants for VIGS experiment |
|---|---|
| fragments from L. serriola referred | L. sativa harboring genetic |
| to as 140:Ch4 + Ch8 | fragments from L.serriola, |
| | referred to as 140:Ch4 + Ch8 |
| fragments from L. serriola referred | L. sativa harboring genetic |
| to as 139:Ch4 + Ch8 | fragments from L.serriola, |
| | referred to as 139:Ch4 + Ch8 |
| fragment from L. serriola referred | L. sativa harboring a |
| to as 139:Ch2 | genetic fragment from |
| | L.serriola, referred to as 139:Ch2 |

After the VIGS silencing, the obtained transformed plants can be subjected to a disease test using Bremia race B1:36. The non-transformed plants are resistant to the pathogen, while it is expected that the plants where the VIGS experiments was successful, will become susceptible.

1. A lettuce plant resistant to Bremia, the lettuce plant comprising a first and a second Bremia-resistance providing genomic fragment from Lactuca serriola, wherein the first Bremia-resistance providing genomic fragment is located on chromosome 4 of the lettuce plant and comprises a nucleic acid sequence selected from the group consisting of SEQ ID No. 1, SEQ ID No. 3, SEQ ID No. 5, SEQ ID No. 7 and SEQ ID No. 9; and the second Bremia-resistance providing genomic fragment is located on chromosome 8 of the lettuce plant, and comprises a nucleic acid sequence selected from the group consisting of SEQ ID No. 11, SEQ ID No. 13, SEQ ID No. 15, SEQ ID No. 17 and SEQ ID No. 19.

2. A lettuce plant resistant to Bremia, the lettuce plant comprises a third and a fourth Bremia-resistance providing genomic fragment from Lactuca serriola, wherein the third Bremia-resistance providing genomic fragment is located on chromosome 4 of the lettuce plant and comprises a nucleic acid sequence selected from the group consisting of SEQ ID No. 31, SEQ ID No. 33, SEQ ID No. 35, SEQ ID No. 37 and SEQ ID No. 39; and the fourth Bremia-resistance providing genomic fragment is located on chromosome 8 of the lettuce plant, and comprises a nucleic acid sequence selected from the group consisting of SEQ ID No. 41, SEQ ID No. 43, SEQ ID No. 45, SEQ ID No. 47 and SEQ ID No. 49.

3. A lettuce plant resistant to Bremia the lettuce plant comprises a fifth Bremia-resistance providing genomic fragment from Lactuca serriola, wherein the fifth Bremia resistance providing fragment is located on chromosome 2 of the lettuce plant and comprises a sequence selected from the group consisting of SEQ ID No. 21, SEQ ID NO. 23, SEQ ID No. 25, SEQ ID No. 27 and SEQ ID No 29.

4. The lettuce plant resistant to Bremia of claim 3, further comprising:
a) a first Bremia-resistance providing genomic fragment selected from the group consisting of SEQ ID No. 1, SEQ ID No. 3, SEQ ID No. 5, SEQ ID No. 7 and SEQ ID No. 9 and a second Bremia-resistance providing genomic fragment selected from the group consisting of SEQ ID No. 11, SEQ ID No. 13, SEQ ID No. 15, SEQ ID No. 17, and SEQ ID No. 19; or
b) a third Bremia-resistance providing genome fragment selected from the group consisting of SEQ ID No. 31, SEQ ID No. 33, SEQ ID No. 35, SEQ ID No. 37 and SEQ ID No. 39 and a fourth Bremia-resistance providing genomic fragment selected from the group consisting of SEQ ID No. 41, SEQ ID No. 43, SEQ ID No. 45, SEQ ID No. 47 and SEQ ID No. 49.

5. The lettuce plant resistant to Bremia according to claim 1, wherein the Bremia-resistance is resistance against races B1:33 to B1: 36 and B1:25 to B1:31.

6. The lettuce plant according to claim 4, wherein:
a) the first and the second Bremia-resistance providing genomic fragments are obtainable, obtained, or are from a lettuce plant, the seeds of which are deposited under deposit number NCIMB 43820;
b) the third and the fourth Bremia-resistance providing genomic fragments are obtainable, obtained or are from a lettuce plant, the seeds of which are deposited under deposit number NCIMB 43819 or NCIMB 43818;
c) the fifth Bremia-resistance providing genomic fragment is obtainable, obtained, or is from a lettuce plant the seeds of which are deposited under deposit number NCIMB 43821 or NCIMB 43818.

7. Seed or progeny of a lettuce plant resistant to Bremia according to claim 4, wherein the seed or progeny comprise in its genome:
a) the first and the second Bremia-resistance providing genomic fragments from Lactuca serriola, or
b) the third and the fourth Bremia-resistance providing genomic fragments from Lactuca serriola, or
c) the fifth Bremia-resistance providing genomic fragment from Lactuca serriola, or
d) the fifth Bremia-resistance providing genomic fragment from Lactuca serriola in combination with:
1) the first and the second Bremia-resistance providing genomic fragments from Lactuca serriola, or
2) the third and the fourth Bremia-resistance providing genomic fragments from Lactuca serriola.

8. An edible part, pollen, egg cell, callus, suspension culture, somatic embryo, clone, embryo or plant part of a lettuce plant resistant to Bremia according to claim 1.

9. A method for identifying a Bremia resistant plant, the method comprising the steps of:
i) isolating or providing the genomic nucleic acid of the plant,
ii) detecting in the isolated genomic nucleic acid:
a first Bremia-resistance providing genomic fragment selected from the group consisting of SEQ ID No. 1, SEQ ID No. 3, SEQ ID No. 5, SEQ ID No. 7 and SEQ ID No. 9 and a second Bremia-resistance providing genomic fragment selected from the group consisting of SEQ ID No. 11, SEQ ID No. 13, SEQ ID No. 15, SEQ ID No. 17, and SEQ ID No. 19, and/or
a third Bremia-resistance providing genome fragment selected from the group consisting of SEQ ID No. 31, SEQ ID No. 33, SEQ ID No. 35, SEQ ID No. 37 and SEQ ID No. 39 and a fourth Bremia-resistance providing genomic fragment selected from the group consisting of SEQ ID No. 41, SEQ ID No. 43, SEQ ID No. 45, SEQ ID No. 47 and SEQ ID No. 49, and/or
a fifth Bremia-resistance providing genomic fragment from Lactuca serriola selected from the group consisting of SEQ ID No. 21, SEQ ID No. 23, SEQ ID No. 25, SEQ ID No. 27, and SEQ ID No. 29.

10. The method according to claim 9, wherein step (i) or step (ii) comprises nucleic acid amplification.

11. (canceled)

12. (canceled)

13. A method of producing a lettuce plant that is resistant to Bremia, wherein the method comprises the step of introgressing:
a) a first Bremia-resistance providing genomic fragment Lactuca serriola selected from the group consisting of SEQ ID No. 1, SEQ ID No. 3, SEQ ID No. 5, SEQ ID No. 7 and SEQ ID No. 9 and a second Bremia-resistance providing genomic fragment Lactuca serriola selected from the group consisting of SEQ ID No. 11, SEQ ID No. 13, SEQ ID No. 15, SEQ ID No. 17, and SEQ ID No. 19, or
b) a third Bremia-resistance providing genome fragment from Lactuca serriola selected from the group consisting of SEQ ID No. 31, SEQ ID No. 33, SEQ ID No. 35, SEQ ID No. 37 and SEQ ID No. 39 and a fourth Bremia-resistance providing genomic fragment from Lactuca serriola selected from the group consisting of SEQ ID No. 41, SEQ ID No. 43, SEQ ID No. 45, SEQ ID No. 47 and SEQ ID No. 49, or
c) a fifth Bremia-resistance providing genomic fragment from Lactuca serriola selected from the group consisting of SEQ ID No. 21, SEQ ID No. 23, SEQ ID No. 25, SEQ ID No. 27, and SEQ ID No. 29 from Lactuca serriola, or
d) a fifth Bremia-resistance providing genomic fragment from Lactuca serriola selected from the group consisting of SEQ ID No. 21, SEQ ID No. 23, SEQ ID No. 25, SEQ ID No. 27 and SEQ ID No. 29 in combination with:
i) a first Bremia-resistance providing genomic fragment Lactuca serriola selected from the group consisting of SEQ ID No. 1, SEQ ID No. 3, SEQ ID No. 5, SEQ ID No. 7 and SEQ ID No. 9 and a second Bremia-resistance providing genomic fragment Lactuca serriola selected from the group consisting of SEQ ID No. 11, SEQ ID No. 13, SEQ ID No. 15, SEQ ID No. 17, and SEQ ID No. 19; or
ii) a third Bremia-resistance providing genome fragment selected from Lactuca serriola selected from the group consisting of SEQ ID No. 31, SEQ ID No. 33, SEQ ID No. 35, SEQ ID No. 37 and SEQ ID No. 39 and a fourth Bremia-resistance providing genomic fragment from Lactuca serriola selected from the group consisting of SEQ ID No. 41, SEQ ID No. 43, SEQ ID No. 45, SEQ ID No. 47 and SEQ ID No. 49,
into a lettuce plant not comprising the first, the second, the third, the fourth or the fifth Bremia resistance providing genomic fragment.

14. The method for providing a Bremia resistant lettuce plant according to claim 13, wherein the Bremia resistance-providing genomic fragment or fragments from Lactuca serriola—are introduced into a lettuce plant, wherein in the Bremia resistant lettuce plant is not exclusively obtained by means of an essentially biological process.

15. The method according to claim 14, wherein the method for providing comprises at least the step of co-transformation with Agrobacterium and/or CRISPR/Cas.

16. (canceled)


SEQUENCE LISTING (50 sequences)

SEQ ID NO: 1 - DNA, 101 bp, Lactuca serriola
attttgattggttgataagaatttcactacaacaccaaatgattctcgacatctagcagtggtagaatttctcgaactcatttttatcgagttttaacaaa

SEQ ID NO: 2 - DNA, 101 bp, Lactuca sativa
attttgattggttgataagaatttcactacaacaccaaatgattctcgacctctagcagtggtagaatttctcgaactcatttttatagagttttaacaaa

SEQ ID NO: 3 - DNA, 101 bp, Lactuca serriola
ctgatttgagatcttgcacactcctaataataacacaatacatggtctaattttgtttttgtttattttgcttctcttggtaccatgttcgtatgcacaca

SEQ ID NO: 4 - DNA, 101 bp, Lactuca sativa
ctgatttgagaccttgcacactcctaataataacacaatacatggtctaactttgtttttgtttattttgcttctcttggtaccatgttcgtatgcacaca

SEQ ID NO: 5 - DNA, 101 bp, Lactuca serriola
caaaaatggccgccagaatagctcgtaactgcaacacaccaaagagtctcacaatcgaatgacacgtagaaaacaattgtttaccgcagtcaaaatttcgc

SEQ ID NO: 6 - DNA, 101 bp, Lactuca sativa
caaaaatggccgccagaatagctcgtaactgcaacacaccaaagagtctcccaatcgaatgacacgtagaaaacaattgtttaccgcagtcaaattttcgc

SEQ ID NO: 7 - DNA, 101 bp, Lactuca serriola
gcaaagaagggtcctgctaaggaaacatgaggatgagaagggcatttatgcctttttctcacccggttttttattgttgttttcttatttgaccttttgga

SEQ ID NO: 8 - DNA, 101 bp, Lactuca sativa
gcaaagaagggtcctgctaaggaaacatgaggatgagaagggcatttatgtctttttctcacccggttttttattgttgttttcttatttgaccttttgga

SEQ ID NO: 9 - DNA, 101 bp, Lactuca serriola
acacaaccaaataataaaataaaataattttcataaattatacttagaggctgtttggatgtactttttaatctcaaaagtgtttttgggaaaagtcttta

SEQ ID NO: 10 - DNA, 101 bp, Lactuca sativa
acacaaccaaataataaaataaaataattttcataaattatacttagagggtgtttggatgtactttttaatctcaaaagtgtttttgggaaaagtcttta

SEQ ID NO: 11 - DNA, 101 bp, Lactuca serriola
ggaaagatttgtgtgctttgattccatttgcaggtatagttttatttcaacattttggttcatgatgtttcaaatgatggttggaacacatgaatttgatt

SEQ ID NO: 12 - DNA, 101 bp, Lactuca sativa
ggaaagatttgtgtgctttgattccatttgcaggtatagttttatttcaaaattttggttcatgatgtttcaaatgatgtttggaacacatgaatttgatt

SEQ ID NO: 13 - DNA, 101 bp, Lactuca serriola
tggatccttgtggtcattttgtgttatatgtttaagatctaaggatccttcgagttttgttcccatgctcaagctttaagtactcatttgccacttctcat

SEQ ID NO: 14 - DNA, 101 bp, Lactuca sativa
tggatccttgtggtcattttgtgttatatgtttaaaatctaaggatccttggagttttgttcccatgctcaagctttaagtactcatttgccacttctcat

SEQ ID NO: 15 - DNA, 101 bp, Lactuca serriola
aaacaacaagaattggtggtttactcacctcccaagtggaattaattaattggttcaagcagaaaagctcaaaaccccagtaattaaattaaataacctaa

SEQ ID NO: 16 - DNA, 101 bp, Lactuca sativa
aaacaacaagaattggtggtttactcacctcccaagtggaattaattaatgggttcaagcagaaaagctcaaaaccccagtaattaaattaaataacctaa

SEQ ID NO: 17 - DNA, 101 bp, Lactuca serriola
attacaagaggattaatgcatataagtttagaatctaacagatttcagtacatggcatgtaagtgcctaaattcgcctgtgtatcaatcagatccatttga

SEQ ID NO: 18 - DNA, 101 bp, Lactuca sativa
attacaagaggattaatgcatataagtttagaatctaacagatttcagtaaatggcatgtaagtgcctaaattcgcctgtgtatcaatcagatccatttga

SEQ ID NO: 19 - DNA, 101 bp, Lactuca serriola
agttagagtttcataccatgcatgagatgcttgttttaagccgtacaccgtcttatccaaaattataacatggtttggaaattcctcgtttacgaaacctg

SEQ ID NO: 20 - DNA, 101 bp, Lactuca sativa
agttagagtttcataccatgcatgagatgcttgttttaagccgtacaccgccttatccaaaagtataacatggtttggaaattcctcgtttacgaaacctg

SEQ ID NO: 21 - DNA, 101 bp, Lactuca serriola
aactgcttgtttttatatatttccctcttgcagatgttttacatggaagaatgagaggttgcttgtggaaagccaccgattacatgtcaaataaagtcttt

SEQ ID NO: 22 - DNA, 101 bp, Lactuca sativa
aactgcttgtttttatatatttccctcttgcagatcttttacatggaagagtgagaggttgcttgtggaaagccaccgattacatgtcaaataaagtcttt

SEQ ID NO: 23 - DNA, 101 bp, Lactuca serriola
agctcttggaggagcatctcacgtctttttgagccaagattgttgcggggaagattggggtgtgaactccctcatttcgggagttcaaggtgtgtagagtt

SEQ ID NO: 24 - DNA, 101 bp, Lactuca sativa
agctcttggaggagcatctcacgtctttttgagccaagattgttgcggggcagattggggtgtgaactccctcatttcgggagttcaaggtgtgtagagtt

SEQ ID NO: 25 - DNA, 101 bp, Lactuca serriola
agaaaaactcccgcatccttgcactttggaggtaaacccttttccaaaatgtttgaaatgttttcactagcttttagtgtttcgttagttttaattttttt

SEQ ID NO: 26 - DNA, 101 bp, Lactuca sativa
agaaaaactcccgcatccttgcactttggaggtaaacccttttccaaaatagttgaaatgttttcactagcttttagtgtttcgttagttttaattttttt

SEQ ID NO: 27 - DNA, 101 bp, Lactuca serriola
tggttaatttttgaaaagcgaaaggcagttaagtgattataattttctaatagattgatcttaacttgtacaaactttcaaataggaaaacccatactcta

SEQ ID NO: 28 - DNA, 101 bp, Lactuca sativa
tggttaatttttgaaaagcgaaaggcagttaagtgattataattttctaaaagattgatcttaacttgtacaaactttcaaataggaaaacccatactcta

SEQ ID NO: 29 - DNA, 101 bp, Lactuca serriola
tggaaactcaaagactgtccattgatttctcttccatagattccttaaactatctaaccccactaactccagttgtgttaggtttggaggattgataagag

SEQ ID NO: 30 - DNA, 101 bp, Lactuca sativa
tggaaactcaaacactgtccattgatttctcttccatagattccttaaacgatctaatcccactaactccagttgtgttaggtttggaggattgataagag

SEQ ID NO: 31 - DNA, 101 bp, Lactuca serriola
ttttaaatttatgaagggcttcatccttagaatgtaatagatatacacaacaaaattagaataatcatttataaagataaccatgtatttcttactactta

SEQ ID NO: 32 - DNA, 101 bp, Lactuca sativa
ttttaaatttatgaagggcttcatccttagaatgtaatagatatacacaataaaattagaataatcatttataaagataaccatgtatttcttactactta

SEQ ID NO: 33 - DNA, 101 bp, Lactuca serriola
cgagacattacatcgattcaggtgagtttcgcctctctgtacccgtgggtggaaggcaccaatgtcagcccactagttttggttatatgttagtagaaggg

SEQ ID NO: 34 - DNA, 101 bp, Lactuca sativa
cgagacattacatcgattcaggtgagtttcgcctctctgtacccgtgggttgaaggcaccaatgtcagcccactagttttggttatatgttagtagaaggg

SEQ ID NO: 35 - DNA, 101 bp, Lactuca serriola
aaaagcatagtattggaatggtggacatgtcggaatattatgttaccccagacaagtgtaggaccaataagactaatcaacatcattttgaagttgtgatt

SEQ ID NO: 36 - DNA, 101 bp, Lactuca sativa
aaaagcatagtattggaatggtggacatgtcggaatattatgttaccccacacaagtgtaggaccaataagactaatcaacatcattttgaagttgtgatt

SEQ ID NO: 37 - DNA, 101 bp, Lactuca serriola
ctgttcattttttgaatttcgtctgaagtttttgacgatgaagaacgaaggatctatctatctgttctaatgttgtttgtgtttaggtgaacaacaaaccc

SEQ ID NO: 38 - DNA, 101 bp, Lactuca sativa
atgttcattttttgaatttcgtctgaagtttttgacgatgaagaacgaagaatctatctatctgttctaatgttgtttgtgtttaggtgaacaataaaccc

SEQ ID NO: 39 - DNA, 101 bp, Lactuca serriola
aaggctggaggggcatacacaactataaaaagcacttctaggcccgaacggatccaaaccgcctgaatagctacataccaatcatgaatcgaaacccgtgt

SEQ ID NO: 40 - DNA, 101 bp, Lactuca sativa
aaggcttgaggggcatacacaactataaaaagcacttctaggcccgaacgaatccaaaccgcctgaattgctacataccaatcatgaatcgaaacccgtgt

SEQ ID NO: 41 - DNA, 101 bp, Lactuca serriola
agattcactaatccatcaatggagaaacacccagaaagatgaagatcaaggtgcttacaacccaatgacgagtttatctgaagaacaagtgatcatcgtcc

SEQ ID NO: 42 - DNA, 101 bp, Lactuca sativa
agattcactaatccatcaatggagaaacacccagaaagatgaagatcaagatgcttacaacccaatgacgagtttatctgaagaacaagtgatcatcgtcc

SEQ ID NO: 43 - DNA, 101 bp, Lactuca serriola
tttcaggtacttctggctctaagggtaaaggcccgacttgatggtgcatcatacactctccaatattccgcactagttgttttggatactttgatcttttg

SEQ ID NO: 44 - DNA, 101 bp, Lactuca sativa
tttcaggtacttctagctctaagggtaaaggcccgacttgatggtgcatcgtacactctccaatattccgcactagttgttttggatactttgatcttttg

SEQ ID NO: 45 - DNA, 101 bp, Lactuca serriola
aagatgctttcaaagcacacaccactcgattccgcattttgtgatttactatgatattgatgatgtatctatacattttgataactttggttttataaaaa

SEQ ID NO: 46 - DNA, 101 bp, Lactuca sativa
aagatgctttcaaagcacacaccactcgattccgcattttgtgatttactctgatattgatgatgtatctatacattttgataactttggttttataaaaa

SEQ ID NO: 47 - DNA, 101 bp, Lactuca serriola
aacacctccaagagccaaaaaggacaaatcatttggacataatcatctaagactcaaaggacctcaagttggggaccaaaatgtgacataaaacacccaaa

SEQ ID NO: 48 - DNA, 101 bp, Lactuca sativa
aacacctccaagagccaaaaaggacaaatcatttggacataatcatctaaaactcaaaggacctcaagttggggaccaaaatgtgacataaaacacccaaa

SEQ ID NO: 49 - DNA, 101 bp, Lactuca serriola
tattaatccattaagacatcacaaatcttgcatggaagggatagaaggaatgagatcacatttttatggttcctaatagctccataatggataaagtttca

SEQ ID NO: 50 - DNA, 101 bp, Lactuca sativa
tattaatccattaagacatcacaaatcttgcatggaagggatagaaggaacgagatcacatttttatggttcctaatagctccataatggataaagtttca